(19) United States
(12) Patent Application Publication
(10) Pub. No.: US 2013/0254939 A1
(43) Pub. Date: Sep. 26, 2013

(54) PLANT TRANSFORMATION METHOD PERFORMED VIA GRAFTING OF ROOTSTOCK AND SCION

(71) Applicants: Takeo Harada, Hirosaki-shi (JP); Atsushi Kasai, Hirosaki-shi (JP); Kaori Yamada, Hirosaki-shi (JP); Songling Bai, Morioka-shi (JP)

(72) Inventors: Takeo Harada, Hirosaki-shi (JP); Atsushi Kasai, Hirosaki-shi (JP); Kaori Yamada, Hirosaki-shi (JP); Songling Bai, Morioka-shi (JP)

(73) Assignee: HIROSAKI UNIVERSITY, Hirosaki-shi, Aomori (JP)

(21) Appl. No.: 13/991,257

(22) PCT Filed: Dec. 6, 2011
(86) PCT No.: PCT/JP2011/078150

§ 371 Date: Jun. 3, 2013

(30) Foreign Application Priority Data
Dec. 6, 2010 (JP) 2010-271452

(51) Int. Cl. C12N 15/113

(52) U.S. Cl. CPC C12N 15/113
USPC 800/285

(57) ABSTRACT

An object of the present invention is to provide a method for transforming a plant by grafting of a rootstock and a scion, and using siRNA for initiating transcriptional gene silencing. The method for transforming a plant by grafting of a rootstock and a scion of the present invention as a means for resolution is characterized in that siRNA for initiating transcriptional gene silencing is produced in a scion, the siRNA produced in the scion is transported to a rootstock by grafting, and the rootstock is transformed by initiating transcriptional gene silencing therein.

### TECHNICAL FIELD

The present invention relates to a method for transforming a plant by grafting of a rootstock and a scion.

### BACKGROUND ART

It is well known to those skilled in the art that as a means for improving plant breeds, a method for transforming a plant by suppressing the expression of a specific target gene is effective. Recently, as one of such methods, gene silencing that inhibits a function of gene expression has been attracting attention. Gene silencing is categorized into transcriptional gene silencing (TGS) that occurs at a gene transcriptional level and post-transcriptional gene silencing (PTGS) that occurs after transcription, and it is known that both transcriptional and post-transcriptional gene silencing can be initiated by siRNA (short interference RNA). SiRNA is a low-molecular weight RNA of 20-25 bp in length and is produced by cleaving a double-strand RNA (dsRNA) formed in a cell by a dicer. A single strand generated by dissociating siRNA by a helicase forms an RNA-induced silencing complex (RISC) and binds to a target mRNA and can cleave this target mRNA. SiRNA initiates PTGS by this function. Further, siRNA induces the methylation of a promoter region of a target gene (RNA-directed DNA Methylation (RdDM)), and also is involved in the modification of a histone protein in the region, etc., and by the remodeling of the region, TGS is initiated. TGS is called epigenetic mutation and it is known that silencing is maintained even after going through somatic cell division or meiosis and inherited to progeny.

SiRNA which is carried into sieve tube (phloem) from companion cell via plasmodesmata is known to be transported over a long distance, and such transport also occurs by grafted plant. By utilizing this property of siRNA, a method in which siRNA for initiating PTGS is produced in a scion, the siRNA produced in the scion is transported to a rootstock by grafting, and the rootstock is transformed by initiating PTGS therein is disclosed in Non-patent document 1. However, there has not been reported any case of using siRNA for initiating TGS, and its function has not been elucidated yet.

### PRIOR ART DOCUMENTS

### Non-Patent Documents

Non-patent document 1: Molnar A. et al., Science 328: 872-875. 2010

### SUMMARY OF THE INVENTION

### Problems that the Invention is to Solve

Accordingly, an object of the present invention is to provide a method for transforming a plant by grafting of a rootstock and a scion, and using siRNA for initiating TGS.

### Means for Solving the Problems

A method for transforming a plant by grafting of a rootstock and a scion according to the present invention made in view of the above problem is characterized in that, as described in claim 1, siRNA for initiating TGS is produced in a scion, the siRNA produced in the scion is transported to a rootstock by grafting, and the rootstock is transformed by initiating TGS therein.

Further, a method described in claim 2 is characterized in that in the method described in claim 1, as the method for producing the siRNA for initiating TGS in the scion, a method for infecting a scion with an Agrobacterium, into which a vector using a CoYMV promoter and capable of producing siRNA containing a sequence homologous to that of a promoter region of a target gene has been introduced, is used.

Further, a method for obtaining a transformant of a plant according to the present invention is characterized in that, as described in claim 3, a rootstock is transformed by the method described in claim 1, and thereafter a regenerated plant from a lateral root from a primary root of the rootstock is obtained as a transformant.

### Effect of the Invention

According to the present invention, a method for transforming a plant by grafting of a rootstock and a scion, and using siRNA for initiating TGS can be provided.

### BRIEF DESCRIPTION OF THE DRAWINGS

FIG. 1 A schematic view showing main parts of constructs of an siRNA producing vector (silencer) and a target gene producing vector (target) in Example.

FIG. 2 A view showing observation results of initiation of TGS in a grafted plant in Example.

FIG. 3 A view showing observation results of initiation of TGS at a branched portion of a lateral root from a primary root of a rootstock in Example.

FIG. 4 A view showing observation results of initiation of TGS at a tip end of a lateral root in Example.

### MODE FOR CARRYING OUT THE INVENTION

The method for transforming a plant by grafting of a rootstock and a scion according to the present invention is characterized in that siRNA for initiating TGS is produced in a scion, the siRNA produced in the scion is transported to a rootstock by grafting, and the rootstock is transformed by initiating TGS therein.

In the present invention, as the method for producing siRNA for initiating TGS in a scion, a method as described below can be exemplified. A vector capable of producing siRNA containing a sequence homologous to that of a promoter region of a target gene is introduced into an Agrobacterium such as an Agrobacterium tumefacience EHA105 strain, and then, a lamina of a plant to be used as a scion is infected with the Agrobacterium carrying the siRNA producing vector by a known method per se, a plant regenerated from a cell in which a desired transformation has occurred by an insertion of T-DNA of the vector is obtained, followed by growing the regenerated plant, and the resulting plant is used as the scion (if necessary, see, for example, Burow, M. D. et al., Plant Mol. Biol. Rep. 8: 124-139. 1990 or Ratchlif, F. CG. et al., Plant Cell 11: 1207-1216. 1999, etc.).

As the siRNA producing vector, a vector having a structure in which an inverted repeat sequence construct comprising a sense strand sequence (which may be a partial sequence) of a promoter region of a target gene and an antisense strand sequence thereof is integrated between a promoter and a terminator can be exemplified (a spacer may be inserted in the inverted repeat sequence construct). In order to efficiently transport siRNA produced in a scion to a rootstock through a sieve tube, as the promoter, a promoter which functions specifically in a companion cell serving as the origin of a sieve tube transport, for example, a CoYMV (Commelina yellow mottole virus) promoter, is desirably used. Incidentally, as the terminator, for example, an NOS terminator which functions as a terminator in a plant body, etc. can be exemplified.

The plant to which the present invention is applied is not particularly limited as long as it is a plant which can be used as either of a rootstock and a scion for grafting. As the grafting method, a method known per se may be employed. According to the present invention, by transporting siRNA by grafting from a scion having a high source strength to a rootstock having a high sink strength, TGS is effectively initiated in the rootstock, whereby the rootstock can be transformed. Since TGS initiated in the rootstock is inherited to progeny, when a regenerated plant is obtained by a culture of a tissue from a lateral root formed by the division of a pericycle cell adjacent to a sieve tube in a primary root of a rootstock, or in case of a plant in which so-called “basal shoot” may appear (e.g., a fruit tree such as a blueberry tree or an apple tree), when a root sucker is obtained as a regenerated plant, such a plant can be grown as an improved breed because it is a transformed plant in which silencing is maintained.

### EXAMPLES

Hereinafter, the present invention will be described in detail with reference to Examples, however, the present invention is not construed as being limited to the following description.

(1) Production of siRNA producing vector for producing siRNA that initiates TGS in scion

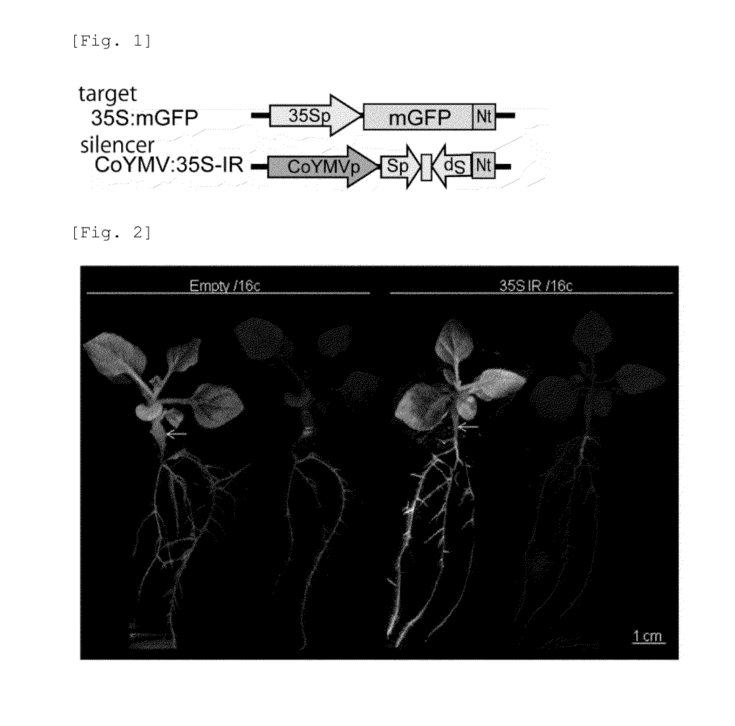

An intron derived from CAT1 (catalase) gene (sequence length: 201 bp, Ohta S. et al., Plant and Cell Physiology 31: 805-813. 1990) was ligated and integrated as a spacer in an inverted repeat sequence construct comprising a region (−32 to −342 bp) of a CaMV35S promoter (Okano Y. et al., Plant Journal 53: 65-77. 2008) and an antisense strand sequence thereof. GUS (beta-glucuronidase) gene at BamHI/SacI sites of a binary vector pE2113-GUS (Mitsuhara I. et al., Plant Cell Physiology 37: 49-59. 1996.) was replaced with the above unit to construct 35S:35S-IR. Subsequently, CoYMVp which is a promoter specifically functioning in a companion cell was amplified by PCR using pCOI (Matsuda, Y. et al., Protoplasma 220: 51-58. 2002) and a fragment at SalI/BamHI sites of the 35S:35 S-IR was replaced with CoYMVp, whereby a target siRNA producing vector (CoYMV:35S-IR) was obtained (see silencer in FIG. 1).

(2) Introduction of siRNA Producing Vector into Agrobacterium

As the Agrobacterium, an Agrobacterium tumefacience EHA105 strain was used. A single colony of the strain was inoculated into a medium obtained by adding an antibiotic (50 mg/L rifampicin) to an LB medium (see Table 1 for the composition thereof), and shaking culture was performed at 28° C. for 24 hours. Then, the strain was subcultured and shaking culture was further performed for 12 hours. Thereafter, centrifugation was performed at 6000 rpm for 10 minutes at 4° C., and the collected bacterial cells were washed with sterile water and 10% glycerol. This bacterial cell pellet was suspended in 1 mL of 10% glycerol. A 40 μL portion of the suspension was mixed with 0.5 to 1.0 μg of the siRNA producing vector produced in (1), and the mixed liquid was transferred to a cuvette. Then, the siRNA producing vector was introduced into the Agrobacterium by electroporation at 20 kV/cm for 6 ms. To the reaction liquid in the cuvette to which a voltage was applied, 1 mL of an LB medium was added, and the resulting mixture was collected in a 1.5 mL tube, and then, the bacterial cells were cultured at 28° C. for 24 hours. The culture solution was applied onto an LB agar medium containing antibiotics (50 mg/L rifampicin and 50 mg/L kanamycin), and the bacterial cells were cultured at 28° C. for 3 days. An obtained colony was cultured in a fresh LB medium, and used for an Agrobacterium infection.

TABLE 1
Composition of LB medium

| Triptone | 10 g/L |
|---|---|
| Yeast extract | 5 g/L |
| NaCl | 5 g/L |
| pH | 7.3 |

(3) Infection of Nicotiana Plant with Agrobacterium Carrying siRNA Producing Vector

To 5 mL of an LB medium, antibiotics (50 mg/L rifampicin and 50 mg/L kanamycin) were added, and the Agrobacterium carrying the siRNA producing vector was cultured overnight at 28° C. The Agrobacterium was subcultured and shaking culture was further performed for 12 hours. Thereafter, centrifugation was performed at 3000 rpm for 20 minutes at room temperature, and the collected bacterial cells were suspended in a suspension medium (see Table 2 for the composition thereof) to give an OD600 of 1.0. A lamina of a Nicotiana benthamiana plant on days 15 after germination, which was aseptically cultivated under light conditions, was subjected to an Agrobacterium infection by being immersed in the thus prepared suspension of the Agrobacterium carrying the siRNA producing vector. Then, a plant regenerated from a cell in which a desired transformation occurred was obtained according to a common procedure.

TABLE 2
Composition of Agrobacterium suspension

| | | |
|---|---|---|
| Magnesium sulfate | 4.314 | mg/L |
| MS vitamin | 10 | mL/L |
| Acetosyringone | 30 | g/L |

### (4) Grafting of Rootstock and Scion

A hypocotyl region (about 5 mm below a cotyledon) of a Nicotiana benthamiana 16C (a green fluorescent protein producing transformant into which a target gene producing vector 35S:mGFP in this Example shown as target in FIG. 1 has been introduced, Jones L. et al., Plant Cell 11: 2291-2301. 1999) plant on days 7 after germination, which was cultivated in MS agar (0.7%) in a greenhouse under light conditions, was cut horizontally with a razor, and the plant body on the side of the root was used as a rootstock. On the other hand, the Nicotiana benthamiana plant on days 7 after germination infected with the Agrobacterium carrying the siRNA producing vector in (3) was also treated in the same manner, and the plant body on the side of the cotyledon was used as a scion. Grafting was performed by bringing the hypocotyl regions of both plant bodies into close contact with each other in a silicone tube (2 mm (length)×0.5 mm (outer diameter)×0.4 mm (inner diameter)). All the operations were aseptically performed under a microscope. The grafted plant was set up using agarose (3 mm cube) in a sterile dish. After 7 days, the tube was removed, and the plant was cultivated in rockwool (Nitto Boseki Co.) using a liquid fertilizer (Otsuka House Nos. 1 and 2, Otsuka Chemical Co.).

### (5) Observation of Initiation of TGS

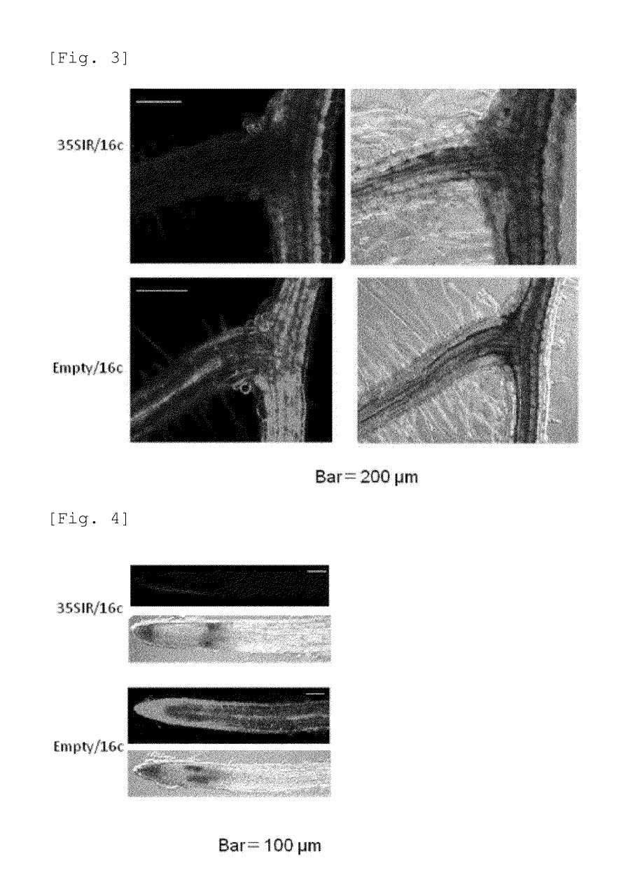

Observation was performed 7 days after the grafting. The results of the observation of the grafted plant under visible light and UV light are shown in FIG. 2 (35SIR/16c, left image: under visible light, right image: under UV light, arrow: grafting point). Incidentally, in FIG. 2, the results of observation of a grafted plant obtained by performing the same procedure using a vector which does not contain the siRNA expression unit under visible light and UV light are also shown (Empty/16c, left image: under visible light, right image: under UV light, arrow: grafting point). Further, a sample of each of the grafted plants was embedded in a 7% low-melting point agarose block, and a section with a thickness of 100 μm was prepared using a vibratome (Series 1500, Leica, St. Louis, Mo.), and then, a branched portion of a lateral root from a primary root of the rootstock and a tip end of the lateral root were observed using a confocal laser scanning microscope (Confocal laser scanning microscopy system FluoVie 1000, Olympus, Tokyo). The results of the observation of the branched portion of the lateral root from the primary root of the rootstock are shown in FIG. 3, and the results of the observation of the tip end of the lateral root are shown in FIG. 4 (right image of FIG. 3 and lower image of FIG. 4: under visible light, left image of FIG. 3 and upper image of FIG. 4: under UV light). As apparent from FIG. 2, in the case of the grafted plant obtained using the siRNA producing vector (35SIR/16c), unlike the case of the grafted plant obtained using the vector which does not contain the siRNA expression unit (Empty/16c), although a slight green fluorescence was observed at around the grafting point, except for this region, green fluorescence was not observed, and therefore, it was found that the siRNA produced in the scion was transported to the rootstock over a long distance through the sieve tube and TGS was effectively initiated in the rootstock. Further, as apparent from FIGS. 3 and 4, in the grafted plant obtained using the siRNA producing vector, it was found that TGS was significantly initiated around the sieve tube of the primary root of the rootstock (it was also confirmed by the observation of initiation of TGS in the cross section of the primary root in another experiment), and that TGS was initiated throughout the lateral root formed from the point. Incidentally, when the presence or absence of green fluorescence was confirmed in a regenerated plant derived from a callus obtained by a tissue culture using a section of the lateral root in which TGS was initiated, green fluorescence was not observed because TGS was inherited to progeny and silencing was maintained. Incidentally, as a comparative experiment, in the case where siRNA produced in a rootstock was transported to a scion by grafting, initiation of TGS was observed in an expanded leaf of the scion, however, a region in which TGS was initiated was not the entire area of the leaf blade, but was limited to a region along the veins. Even when cutting of a stem diagonally was performed for increasing the sink strength of an axillary bud, initiation of TGS was not observed in the entire area of the leaf blade. Accordingly, it was found that initiation of TGS in a rootstock by transporting siRNA produced in a scion to the rootstock by grafting is more effective than initiation of TGS in a scion by transporting siRNA produced in a rootstock to the scion by grafting, and also is advantageous for obtaining a transformed plant in which silencing is maintained.

### INDUSTRIAL APPLICABILITY

The present invention has an industrial applicability in that a method for transforming a plant by grafting of a rootstock and a scion, and using siRNA for initiating TGS can be provided.

1. A method for transforming a plant by grafting of a rootstock and a scion, characterized in that siRNA for initiating transcriptional gene silencing is produced in a scion, the siRNA produced in the scion is transported to a rootstock by grafting, and the rootstock is transformed by initiating transcriptional gene silencing therein.

2. A method according to claim 1, characterized in that as the method for producing the siRNA for initiating transcriptional gene silencing in the scion, a method for infecting a scion with an Agrobacterium, into which a vector using a CoYMV promoter and capable of producing siRNA containing a sequence homologous to that of a promoter region of a target gene has been introduced, is used.

3. A method for obtaining a transformant of a plant, characterized in that a rootstock is transformed by the method according to claim 1, and thereafter a regenerated plant from a lateral root from a primary root of the rootstock is obtained as a transformant.
